(19) United States
(12) Patent Application Publication
(10) Pub. No.: US 2025/0134867 A1
(43) Pub. Date: May 1, 2025

(54) METHOD FOR MODULATING LIPID METABOLISM, AMELIORATING NON-ALCOHOLIC FATTY LIVER DISEASE AND METABOLIC SYNDROME BY USING 6-METHOXYBENZOXAZOLINONE

(71) Applicant: Taichung District Agricultural Research and Extension Station, Ministry of Agriculture, Chang-Hwa County (TW)

(72) Inventors: Yu-Hsin Chen, Chang-Hwa County (TW); Cheng Huang, Chang-Hwa County (TW); Ching-Kuo Lee, Chang-Hwa County (TW); Wei-Chung Chiou, Chang-Hwa County (TW)

(21) Appl. No.: 18/524,700

(22) Filed: Nov. 30, 2023

(30) Foreign Application Priority Data
Nov. 1, 2023 (TW) 112142118

(51) Int. Cl. A61K 31/423; A61P 1/16; A61P 13/12

(52) U.S. Cl. CPC A61K 31/423; A61P 1/16; A61P 13/12

(57) ABSTRACT

The present disclosure provides a method for modulating lipid metabolism, ameliorating non-alcoholic fatty liver disease and metabolic syndrome by using 6-methoxybenzoxazolinone. The 6-methoxybenzoxazolinone of the present disclosure achieves the effect of modulating lipid metabolism, ameliorating non-alcoholic fatty liver disease and metabolic syndrome through various efficacy experiments.

### CROSS-REFERENCE TO RELATED APPLICATION

This application claims priority of Taiwan patent application No. 112142118, filed on Nov. 1, 2023, the content of which is incorporated herein in its entirety by reference.

### BACKGROUND OF THE INVENTION

### 1. Field of the Invention

The present invention relates to a method for modulating lipid metabolism, ameliorating non-alcoholic fatty liver disease and metabolic syndrome by using 6-methoxybenzoxazolinone (6-MBOA).

### 2. The Prior Art

Non-alcoholic fatty liver disease (NAFLD) refers to the accumulation of fatty deposits in the liver, not necessarily related to alcohol consumption. It is the predominant liver disorder in developed nations. Non-alcoholic steatohepatitis (NASH), the most severe manifestation of NAFLD, can result in liver inflammation, fibrosis, cirrhosis, chronic liver failure, and hepatocellular carcinoma (HCC). Currently, there are no approved treatments for NASH or NAFLD. Therefore, there is an unmet need for new treatments for NAFLD and NASH.

Metabolic syndromes refer to the aggregation of cardiovascular disease risk factors at the physiological metabolic level, including fat accumulation, excessive fat synthesis, hypertension, hyperlipidemia, hyperglycemia, and obesity. There is a positive relationship between body fat production and obesity. When fat is excessively produced in the body or the fat metabolism rate slows down, it is easy to cause fat to accumulate in the liver and form fatty liver. Excess fat would also accumulate in adipose tissue, causing obesity. When fatty liver occurs, it is easy to cause liver damage, thereby increasing the liver index in the blood.

Currently, clinical drug treatments for NAFLD and metabolic syndrome offer limited efficacy and often come with severe side effects, leading many patients to discontinue treatment. Moreover, existing drugs only provide temporary relief from symptoms without addressing the underlying issue. Therefore, developing a novel medication capable of effectively mitigating non-alcoholic fatty liver disease and metabolic syndrome, while also regulating lipid metabolism, stands as a crucial challenge that this invention aims to address. Adenosine 5′-monophosphate-activated protein kinase (AMPK) serves as a vital energy sensor, crucial for regulating cellular energy metabolism. Hence, activating AMP kinase presents a promising approach for treating NAFLD.

In order to solve the above-mentioned problems, those skilled in the art urgently need to develop a novel composition for ameliorating non-alcoholic fatty liver disease and metabolic syndrome, and modulating lipid metabolism for the benefit of a large group of people in need thereof.

### SUMMARY OF THE INVENTION

A primary objective of the present invention is to provide a method for modulating lipid metabolism, comprising administering to a subject in need thereof a composition comprising an effective amount of 6-methoxybenzoxazolinone (6-MBOA).

According to an embodiment of the present invention, the 6-MBOA downregulates de novo lipogenesis in the subject.

According to an embodiment of the present invention, the 6-MBOA upregulates lipolysis and fatty acid beta-oxidation in the subject.

According to an embodiment of the present invention, the 6-MBOA produces no concomitant cytotoxicity to immortalized primary human hepatocytes at 25-400 μM.

According to an embodiment of the present invention, the 6-MBOA mitigates lipid accumulation in the subject.

According to an embodiment of the present invention, the 6-MBOA increases adenosine 5′-monophosphate-activated protein kinase (AMPK) phosphorylation in the subject.

According to an embodiment of the present invention, the lipid metabolism is modulated by regulating acetyl-CoA carboxylase (ACC) phosphorylation, and the 6-MBOA simultaneously downregulates expression level of fatty acid synthase (FAS), and upregulates expression levels of adipose triglyceride lipase (ATGL) and carnitine palmitoyl-transferase 1 (CPT1).

Another objective of the present invention is to provide a method for improving non-alcoholic fatty liver disease (NAFLD) and metabolic syndrome, comprising administering to a subject in need thereof a composition comprising an effective amount of 6-methoxybenzoxazolinone (6-MBOA).

According to an embodiment of the present invention, the 6-MBOA prevents body weight gain in the subject.

According to an embodiment of the present invention, the 6-MBOA ameliorates adipose tissue hypertrophy and dyslipidemia in the subject.

According to an embodiment of the present invention, the 6-MBOA improves glucose homeostasis, glucose tolerance, and insulin resistance (HOMA-IR) in the subject.

According to an embodiment of the present invention, the 6-MBOA improves high fat diet (HFD)-induced fat deposition and inflammation in liver.

According to an embodiment of the present invention, the 6-MBOA ameliorates HFD-induced renal damage.

According to an embodiment of the present invention, the composition is a pharmaceutical composition, a food composition, or an external product composition.

According to an embodiment of the present invention, the pharmaceutical composition is in a dosage form for oral administration.

According to an embodiment of the present invention, the pharmaceutical composition is in a dosage form for parenteral administration.

According to an embodiment of the present invention, the pharmaceutical composition further comprises a pharmaceutically acceptable excipient, carrier, auxiliary and/or food additive.

According to an embodiment of the present invention, the composition has a dosage form of powder, granule, solution, gel, or paste.

In summary, the 6-MBOA of the present invention has been proven that it can effectively modulate lipid metabolism, ameliorate non-alcoholic fatty liver disease, blood glucose homeostasis, and metabolic syndrome through various efficacy experiments.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows effects of 6-methoxybenzoxazolinone (6-MBOA) (eepresented by CX in the figure) on the viability of HuS-E/2 cells. HuS-E/2 cells were treated with CX at 25-1600μ M for 24 hours, and 3-(4,5-dimethylthiazol-2-yl)-2,5-diphenyltetrazolium bromide (MTT) assays were performed to determine the viability at the concentrations indicated. Data (n=3) are shown as the mean±standard error of the mean (SEM). One-way ANOVA with Dunnett's multiple comparisons test was performed to determine the statistical significance of parametric data between a control group and the other groups, indicated by asterisks (*, p<0.05; **, p<0.01; ***, p<0.001).

FIGS. 2A and 2B show effects of CX on lipid accumulation in oleic acid (OA)-induced HuS-E/2 cells. (2A) Micrographs of HuS-E/2 cells treated with or without OA and CX. OA was treated at 0.1 mM and CX at the concentrations indicated. The scale bar is 100 μm. Micrographs are at 200× magnification. (2B) Lipid content of HuS-E/2 cells treated with or without OA and CX. OA was treated at 0.1 mM and CX at the concentrations indicated. Data (n=3) are shown as the mean±SEM. One-way ANOVA with Tukey's multiple comparisons test was performed to determine the statistical difference between each group with every other group, where groups with different letters are of statistical significance (p<0.05) and those with the same letters are of statistical insignificance (p≥0.05).

FIGS. 3A and 3B show effects of CX on OA-induced phosphorylation of adenosine 5′-monophosphate-activated protein kinase (AMPK) and acetyl-CoA carboxylase (ACC) in HuS-E/2 cells. Western blot analysis was used to determine the phosphorylation of adenosine 5′-monophosphate-activated protein kinase (AMPK to pAMPK), the phosphorylation of acetyl-CoA carboxylase (ACC1 to pACC1), inhibition of fatty acid synthase (FAS) levels, and upregulation of levels of adipose triglyceride lipase (ATGL) and carnitine palmitoyl-transferase 1 (CPT1). Data (n=3) are shown as the mean±SEM. One-way ANOVA with Tukey's multiple comparisons test was performed to determine the statistical difference between each group with every other group, where groups with different letters are of statistical significance (p<0.05) and those with the same letters are of statistical insignificance (p≥0.05).

FIGS. 4A-4C show the effect of CX on body weight, body weight gain, food intake, and food efficiency ratio in high fat diet (HFD)-induced obese mice. (4A) Body weight gain during the study. (4B) Food intake. (4C) Food efficiency ratio (FER) is body weight gain (g)/food intake (g). Data are shown as means±SEM (n=8 per group). Graph bars labeled with different letters above represent statistically significant results (P<0.05), based on Tukey post hoc tests one-way ANOVA analysis, whereas bars with the same letter correspond to results that show no statistically significant differences. ND represents normal diet group, and EGCG represents epigallocatechin gallate.

FIGS. 5A-5G show the effect of CX on fat deposition and serum lipid levels in HFD-induced obese mice. (5A) Hematoxylin and eosin (H&E) staining of adipocytes in the epididymal white adipose tissue (eWAT). (5B) The weight of eWAT. (5C) The adipocyte diameters. The level of serum (5D) triglyceride (TG), (5E) total cholesterol (TC), (5F) high-density lipoprotein-cholesterol (HDL-C), and (5G) low-density lipoprotein-cholesterol (LDL-C). The scale bar is 100 μM. Data are shown as means±SEM. Graph bars labeled with different letters above represent statistically significant results (P<0.05) based on Tukey post hoc tests one-way ANOVA analysis, whereas bars with the same letter correspond to results that show no statistically significant differences. In the case where two letters are present above the bars, each letter should be compared separately with the letters of the other bars to determine whether the results show statistically significant differences.

FIGS. 6A-6D show the effect of CX on fasting blood glucose, intraperitoneal glucose tolerance, and insulin resistance in HFD-induced obese mice. (6A) After feeding HFD-induced obese mice for 6 weeks, the fasting blood glucose level gradually increased (### indicates that the difference between the HFD group and the control group is extremely significant; p<0.001). However, after drug (CX) intervention, normal fasting blood glucose levels can be maintained from 0 to 12 weeks (*** indicates that the difference between the CX group and HFD is extremely significant; p<0.001). (6B) Area under the curve (AUC) of intraperitoneal glucose tolerance test (IPGTT). Glucose was injected intraperitoneally at a concentration of 1.0 g/kg body weight, and glucose concentration was measured by tail vein sampling. Data are shown as means±SEM. Fasting glucose differences in FIG. 6A were analyzed using unpaired one-tailed Student's t-test (ND vs. HFD, *** p<0.001, HFD vs. drug, *** p<0.001). Graph bars labeled with different letters above represent statistically significant results (P<0.05) based on Tukey post hoc tests one-way ANOVA analysis, whereas bars with the same letter correspond to results that show no statistically significant differences. In the case where two letters are present above the bars, each letter should be compared separately with the letters of the other bars to determine whether the results show statistically significant differences. (6C) Fasting insulin concentration. (6D) Homeostasis Model Assessment-Insulin Resistance (HOMA-IR) index. The calculation method of HOMA-IR is [fasting insulin concentration (mU/L)× fasting glucose concentration (mmol/L)]/22.5. Data (n=8) are expressed as mean±standard error. ND: normal diet; HFD: high-fat diet; EGCG: epigallocatechin gallate. Different letters (for example: a, b) indicate statistically significant differences (p<0.05).

FIGS. 7A-7H show the effect of CX on fat deposition and inflammation in the livers of HFD-induced obese mice. (7A) Liver weight. (7B) Hepatic triglyceride level. (7C) Hepatic cholesterol level. (7D) H&E staining of transverse liver sections (original magnification×200). Serum levels of the hepatic injury markers (7E) Glutamic oxaloacetic transaminase (GOT) and (7F) Glutamic-pyruvic transaminase (GPT). The serum levels of the liver biochemical markers are based on serum albumin (ALB). Obesity would increase the ALB value, and liver and kidney disease would decrease the ALB value. (7G) albumin (ALB). (7H) Ratio of liver weight and body weight. The scale bar is 100 μM. Graph bars labeled with different letters above represent statistically significant results (P<0.05) based on Tukey post hoc tests one-way ANOVA analysis, whereas bars with the same letter correspond to results that show no statistically significant differences. In the case where two letters are present above the bars, each letter should be compared separately with the letters of the other bars to determine whether the results show statistically significant differences.

FIGS. 8A and 8B show the effect of CX on renal function of HFD-induced obese mice. The serum levels of the kidney function markers (8A) creatinine, CRE and (8B) uric acid, UA. Data are shown as means±SEM. Graph bars labeled with different letters above represent statistically significant results (P<0.05) based on Tukey post hoc tests one-way ANOVA analysis, whereas bars with the same letter correspond to results that show no statistically significant differences. In the case where two letters are present above the bars, each letter should be compared separately with the letters of the other bars to determine whether the results show statistically significant differences.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of +20%, preferably within +10%, and most preferably within +5%.

Unless otherwise stated in the context, “a”, “the” and similar terms used in the specification (especially in the following claims) should be understood as including singular and plural forms.

According to the present invention, 6-methoxybenzoxazolinone (6-MBOA) (CX) (chemical formula C₈H₇NO₃) is a lactam compound in grass plants. The present invention is confirmed that CX has effects on anti-lipid synthesis, promotion of lipolysis, anti-diabetic and AMPK and ACC protein kinase regulatory activities.

According to the present invention, the pharmaceutical composition can be manufactured to a dosage form suitable for parenteral or oral administration, using techniques well known to those skilled in the art, including, but not limited to, injection (e.g., sterile aqueous solution or dispersion), sterile powder, tablet, troche, lozenge, pill, capsule, dispersible powder or granule, solution, suspension, emulsion, syrup, elixir, slurry, and the like.

The pharmaceutical composition according to the present invention may be administered by a parenteral route selected from the group consisting of: intraperitoneal injection, subcutaneous injection, intraepidermal injection, intradermal injection, intramuscular injection, intravenous injection, intralesional injection, sublingual administration, and transdermal administration.

The pharmaceutical composition according to the present invention can comprise a pharmaceutically acceptable carrier which is widely used in pharmaceutical manufacturing technology. For example, the pharmaceutically acceptable carrier can comprise one or more reagents selected from the group consisting of solvent, emulsifier, suspending agent, decomposer, binding agent, excipient, stabilizing agent, chelating agent, diluent, gelling agent, preservative, lubricant, absorption delaying agent, liposome, and the like. The selection and quantity of these reagents fall within the scope of the professional literacy and routine techniques of those skilled in the art.

According to the present invention, the pharmaceutically acceptable carrier comprises a solvent selected from the group consisting of water, normal saline, phosphate buffered saline (PBS), sugar solution, aqueous solution containing alcohol, and combinations thereof.

According to the present invention, the procedure of statistical analysis in Examples 1-3 is as follows. Data were analyzed and plotted with GraphPad Prism 7.03 (GraphPad, USA). One-way ANOVA with Dunnett's multiple comparisons test was performed to determine the statistical significance of parametric data between a control group and the other groups, indicated by asterisks (*, p<0.05; **, p<0.01; ***, p<0.001). One-way ANOVA with Tukey's multiple comparisons test was performed to determine the statistical difference between each group with every other group, where groups with different letters are of statistical significance (p<0.05) and those with the same letters are of statistical insignificance (p≥0.05).

According to the present invention, the procedure of statistical analysis in Examples 4-8 is as follows. Data obtained from all experiments are shown as means ±standard error of the mean (SEM). Differences in body weight and fasting glucose were assessed using the unpaired one-tailed Student's t-test. Data sets that involved more than two groups were assessed by analysis of variance (ANOVA) using Tukey post hoc tests. A P value of 0.05 was considered statistically significant. In the figures, the data with different superscript letters are significantly different based on post hoc ANOVA statistical analysis.

### Example 1

### 6-methoxybenzoxazolinone (6-MBOA) (CX) had little cytotoxicity in HuS-E/2 cells

To determine the cytotoxicity of CX, HuS-E/2 cells were incubated with CX at the concentrations indicated for 24 hours. The CX stock solution was 200 mM in dimethyl sulfoxide (DMSO; Cat. 15578544, J. T. Baker, USA), and stored at −20° C.

The procedure regarding cell culture of immortalized primary human hepatocytes HuS-E/2 cells is as follows. HuS-E/2 cell line, kindly provided by Dr. Shimotohno (Kyoto University, Japan), were maintained in primary hepatocyte medium (PH medium), containing 20 mM HEPES, 10% fetal bovine serum, 15 μg/mL L-proline, 0.25 μg/mL insulin, 50 nM dexamethasone, 44 mM NaHCO₃, 10 mM nicotinamide, 5 ng/ml EGF, 0.1 mM Asc-2P, 100 IU/mL penicillin, 100 μg/mL streptomycin, 10 μg/mL gentamicin, and 1 μg/mL plasmocin in high-glucose Dulbeco's modified Eagle medium (DMEM). Cells were incubated at 37° C. in an incubator supplied with 5% CO₂.

3-(4,5-dimethylthiazol-2-yl)-2,5-diphenyltetrazolium bromide (MTT) (Cat. M5655) (Sigma-Aldrich, USA) stock solution was 5 mg/mL in 1×phosphate-buffered saline (PBS), and stored at −20° C.

The procedure regarding cell viability is as follows. HuS-E/2 cells were seeded at 4.5×10⁴ cells/well in a 96-well plate and were incubated with 0-1600 μM CX for 24 h. MTT was added to each well to a final concentration of 0.5 μg/mL and was incubated at 37° C. for an hour before removal. MTT-formazan crystals formed by metabolically viable cells were dissolved in 200 μL of DMSO for absorbance detection at 550 nm using a SPARK® Multimode microplate reader from TECAN, Switzerland. The 50% cytotoxic concentration (CC₅₀) was calculated using the normalized response (variable slope) model given in GraphPad Prism 7.03 (USA).

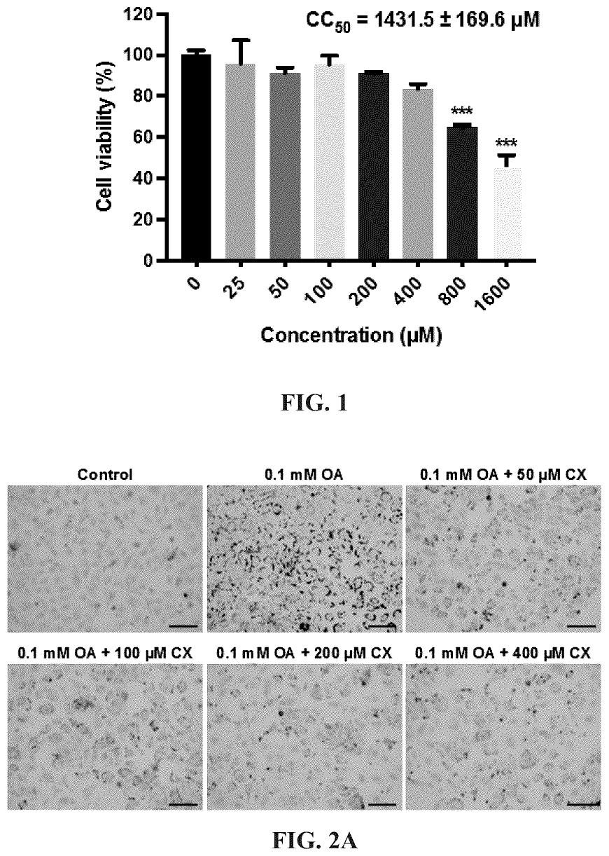

FIG. 1 shows effects of CX on the viability of HuS-E/2 cells. HuS-E/2 cells were treated with CX at 25-1600 μM for 24 hours, and MTT assays were performed to determine the viability at the concentrations indicated. Data (n=3) are shown as the mean±standard error of the mean (SEM). One-way ANOVA with Dunnett's multiple comparisons test was performed to determine the statistical significance of parametric data between a control group and the other groups, indicated by asterisks (*, p<0.05; **, p<0.01; ***, p<0.001).

As shown in FIG. 1, CX had a CC₅₀ value of 1431.5±169.6 μM. In particular, CX at 25-400 μM had no significant effect on the viability of HuS-E/2 cells. Taken together, treating CX at up to 400 μM did not inflict cytotoxicity in HuS-E/2 cells.

### Example 2

### CX Mitigated Lipid Accumulation in Oleic Acid (OA)-Induced HuS-E/2 Cells

To evaluate the effect of CX on lipid accumulation, HuS-E/2 cells were incubated with or without 0.1 mM OA (Cat. O1383) in the presence or absence of CX for 18 hours. The OA stock solution was 3.15 M in DMSO, and stored at −20° C.

The procedure regarding oil red O staining is as follows. Oil Red O stain (Cat. O0625) were from Sigma-Aldrich, USA. The Oil Red O stain stock solution was 3 mg/mL in isopropanol, and stored at room temperature. For microscopic observation, cells were fixed in 4% paraformaldehyde in phosphate-buffered saline (PBS) for 30 min at room temperature, stained with Oil Red O stain working solution (60% Oil Red O stock solution and 40% distilled water) for 1 h at room temperature, and then rinsed with water. Micrographs were captured under a microscope. For quantitative analysis of cellular lipids, cells were washed three times with ice-cold PBS, fixed with 10% formalin for 1 h, washed, stained with Oil Red O solution for 1 h at room temperature, and washed with water to remove excess dye. Cell-bound Oil Red O stain was dissolved in isopropanol at room temperature for 5 min with shaking. The absorbance at 510 nm was measured in a spectrophotometer. The relative lipid content is calculated by setting the readouts of those receiving vehicles to 0% and those receiving free fatty acids alone to 100%.

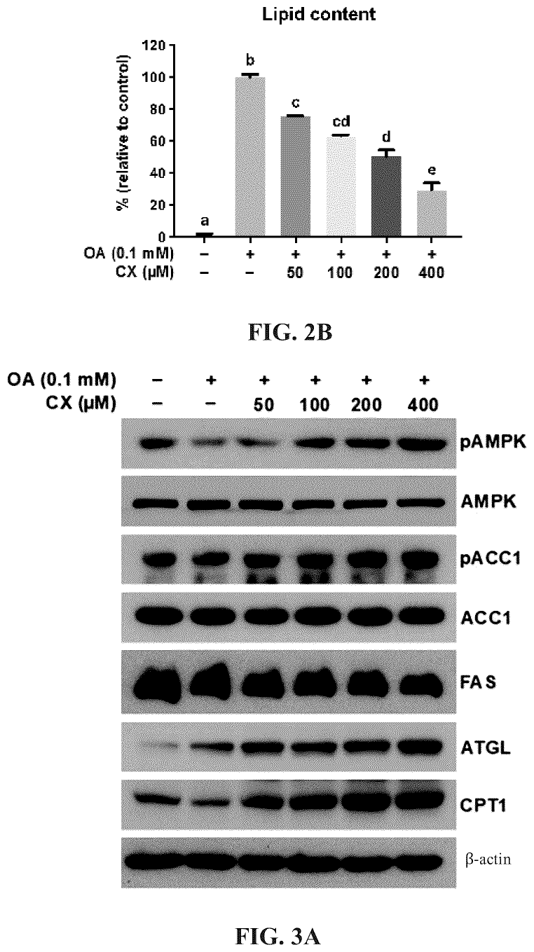

FIGS. 2A and 2B show effects of CX on lipid accumulation in OA-induced HuS-E/2 cells. (2A) Micrographs of HuS-E/2 cells treated with or without OA and CX. OA was treated at 0.1 mM and CX at the concentrations indicated. The scale bar is 100 μm. Micrographs are at 200× magnification. (2B) Lipid content of HuS-E/2 cells treated with or without OA and CX. OA was treated at 0.1 mM and CX at the concentrations indicated. Data (n=3) are shown as the mean±SEM. One-way ANOVA with Tukey's multiple comparisons test was performed to determine the statistical difference between each group with every other group, where groups with different letters are of statistical significance (p<0.05) and those with the same letters are of statistical insignificance (p≥0.05).

As shown in FIG. 2A, HuS-E/2 cells treated with 0.1 mM OA alone manifested lipid accumulation, compared with the control. Meanwhile, CX reduced lipid accumulation in a dose-dependent manner in OA-induced HuS-E/2 cells. On the other hand, the lipid content of HuS-E/2 cells was determined in parallel. As shown in FIG. 2B, CX diminished the lipid content in OA-induced HuS-E/2 cells, corresponding to the observation in micrographs. Taken together, CX ameliorated lipid accumulation in OA-induced HuS-E/2 cells.

### Example 3

### CX Increased AMPK Phosphorylation to Downregulate De Novo Lipogenesis and Upregulate Lipolysis and Fatty Acid Beta-Oxidation in OA-Induced HuS-E/2 Cells

To determine how CX improved lipid accumulation, signaling pathways related to lipid metabolism were investigated. Adenosine 5′-monophosphate-activated protein kinase (AMPK) is an energy sensor that plays a key role in regulating cellular energy metabolism.

The procedure regarding Western blot analysis is as follows. For western blot analysis, an equal amount of protein from each sample was resolved by sodium dodecyl sulfate polyacrylamide gel electrophoresis (SDS-PAGE) and then transferred to polyvinylidene fluoride (PVDF) membranes. Primary antibodies against AMPK (Cat. GTX103487), adipose triglyceride lipase (ATGL) (Cat. GTX109941), carnitine palmitoyl-transferase 1 (CPT1) (Cat. GTX114337), and β-actin (Cat. GTX109639) were from GeneTex (Taiwan), and those against pAMPK (Cat. AP0432), phospho acetyl-CoA carboxylase (pACC) (Cat. AP0298), ACC (Cat. A15606), and fatty acid synthase (FAS) (Cat. A21182) was from ABclonal (China). Horseradish peroxidase (HRP)-conjugated secondary antibodies were from Jackson ImmunoResearch Laboratories, Inc., USA. WesternBright® ECL kits (Advansta Inc., USA) were used for protein visualization. Levels of protein expression were quantified by densitometric analyses.

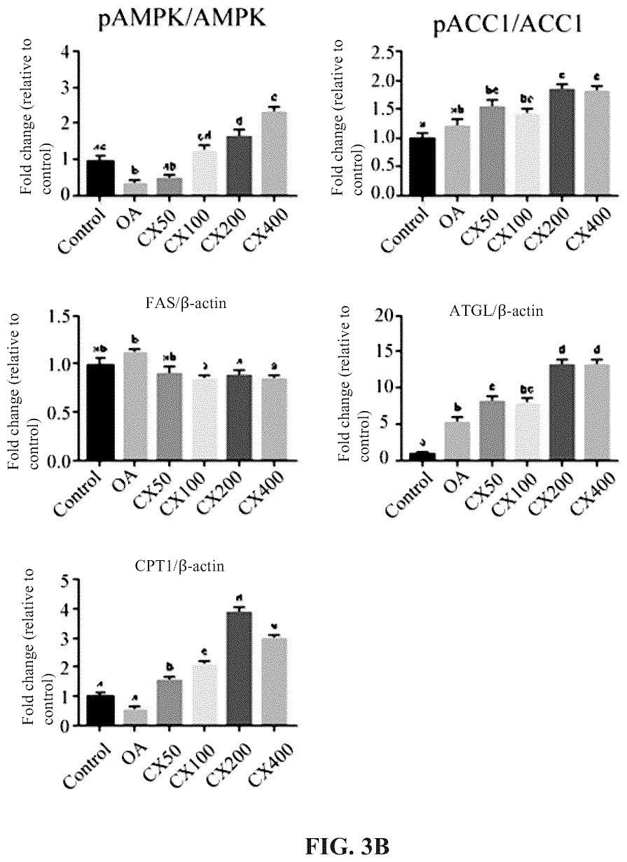

FIG. 3A shows effects of CX on the expression of AMPK, ACC and lipid metabolism-related enzymes in OA-induced HuS-E/2 cells. Protein expression levels of pAMPK, AMPK, pACC1, ACC1, FAS, ATGL, and CPT1 were determined by western blot analysis. Data (n=3) are shown as the mean±SEM. One-way ANOVA with Tukey's multiple comparisons test was performed to determine the statistical difference between each group with every other group, where groups with different letters are of statistical significance (p<0.05) and those with the same letters are of statistical insignificance (p≥0.05).

As shown in FIG. 3B, treating with OA at 0.1 mM alone decreased AMPK phosphorylation significantly, compared with the control. Meanwhile, treating CX at 100-400 μM led to a marked increase in AMPK phosphorylation in OA-induced HuS-E/2 cells. Regarding proteins related to lipid accumulation, acetyl-CoA carboxylase (i.e., ACC1 and ACC2) is a rate-limiting enzyme in lipogenesis. ACC and fatty acid synthase (FAS) are involved in the biosynthesis of long-chain saturated fatty acids. Phosphorylation of ACC enzyme (conversion of ACC into pACC) would reduce the activity of ACC, thereby reducing fatty acid synthesis. Corresponding to upregulated AMPK phosphorylation, CX at 50-400 μM decreased the activity of ACC1 by increasing ACC1 phosphorylation, and CX at 100-400 μM downregulated FAS expression. The above two simultaneously inhibit the production and synthesis of fatty acids. Contrary to ACC1 and FAS, adipose triglyceride lipase (ATGL) is associated with the catalysis and catabolism of lipid droplets and carnitine palmitoyltransferase 1 (CPT1) is engaged in fatty acid beta-oxidation. Consistently, CX at 50-400 μM increased ATGL expression substantially, and CX at 50-400 μM elevated CPT1 levels. Taken together, CX ameliorated lipid accumulation by downregulating lipogenic enzymes and upregulating enzymes that regulate lipolysis and fatty acid β-oxidation in HuS-E/2 cells. Specifically, CX modulated cellular energy homeostasis through AMPK phosphorylation. Correspondingly, CX increased ACC1 phosphorylation and decreased FAS expression to reduce fatty acid synthesis, while promoting ATGL and CPT1 expressions to promote fatty acid catabolism

### Example 4

Intervention with CX Prevented Body Weight Gain and Improved the Food Efficiency Ratio in High Fat Diet (HFD)-Induced Obese Mice

To determine the effect of CX (Sigma, Inc., USA) on obesity and non-alcoholic fatty liver disease (NAFLD) in vivo, we established an experimental approach using HFD-induced obese C₅₇BL/6J mice. CX was suspended in 0.5% methylcellulose solution for oral administration.

The animals used in the examples are described below. 5-week-old male C57BL/6J mice were purchased from The National Laboratory Animal Center, Taipei, Taiwan and were maintained in a temperature-controlled room on a 12-h light-dark cycle. They were housed with four per cage and had free access to food and drinking water. Mice fed with a standard diet and adapted to the environment for 1 week were subsequently divided randomly into five groups. The ND group (n=8) continued on the same diet, whereas the other four groups (n=8 per group) were switched to the HFD (494 kcal/100 g, 45% energy as fat; TestDiet Inc., USA). Among the four HFD groups, three groups started to receive CX at 10 mg/kg/day (CX10 group), CX at 30 mg/kg/day (CX30 group), and epigallocatechin gallate (EGCG) (Sigma, Inc., USA) at 30 mg/kg/day (EGCG30 group) by oral gavage. EGCG was suspended in 0.5% methylcellulose solution for oral administration. Food consumption and weight gain were measured daily and weekly, respectively. After 12 weeks, all mice were sacrificed. Serum samples, liver tissue, epididymis adipose tissue and feces were harvested for further analysis. The experimental protocol was approved by the Animal Research Committee of the National Yang Ming Chiao Tung University (IACUC no. 1091013), and all procedures followed The Guide for the Care and Use of Laboratory Animals (NIH publication, 85-23, revised 1996) and the guidelines of the Animal Welfare Act, Taiwan.

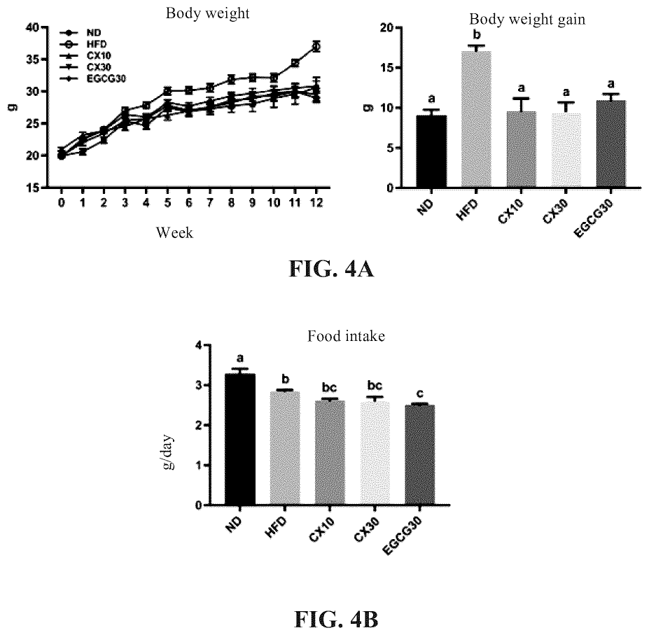

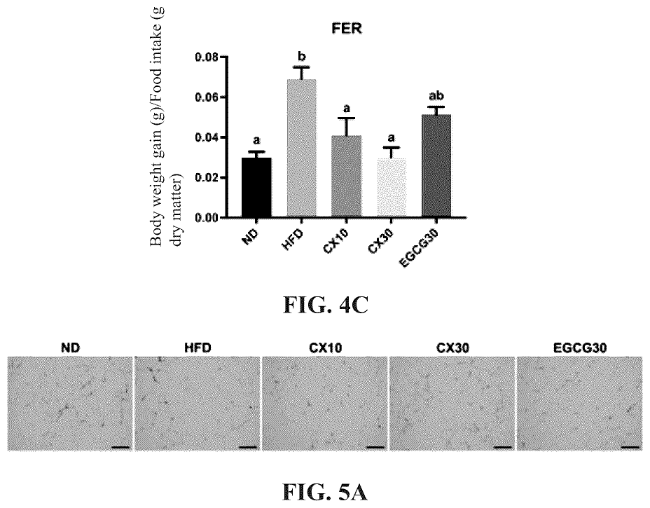

FIGS. 4A-4C show the effect of CX on body weight, body weight gain, food intake, and food efficiency ratio in high fat diet (HFD)-induced obese mice. (4A) Body weight gain during the study. (4B) Food intake. (4C) Food efficiency ratio (FER) is body weight gain (g)/food intake (g). Data are shown as means ±SEM (n=8 per group). Graph bars labeled with different letters above represent statistically significant results (P<0.05), based on Tukey post hoc tests one-way ANOVA analysis, whereas bars with the same letter correspond to results that show no statistically significant differences.

Twelve weeks of HFD feeding resulted in significantly increased body weight and food efficiency ratio (FER) compared with ND control mice (FIGS. 4A and 4C). EGCG was used as a drug control. As shown in FIG. 4A, CX and EGCG significantly prevented body weight gain. Furthermore, the FER of the CX10 and CX30 groups were greatly reduced with almost equal food intake compared to the HFD group (FIGS. 4B and 4C), which means a less body weight gain when eating the same weight of food, after CX treatment.

### Example 5

Adipose Tissue Hypertrophy and Dyslipidemia were Ameliorated in High Fat Diet-Induced Obese Mice after CX Intervention

A well-known feature of metabolic syndrome is increase of lipid accumulation in the trunk region, which causes excessive visceral fat deposition. In order to confirm the rising body weight gain was truly an increase of fat mass, we isolated the epididymal white adipose tissue (eWAT) after dissection of the mice.

The procedure regarding immunohistochemical tissue characterization is as follows. During dissection of the mice, epididymis adipose tissue and liver were isolated, weighed, and subsequently fixed in 10% paraformaldehyde in PBS. After overnight fixation, tissues were embedded in paraffin for hematoxylin and eosin (H&E) staining. All specimens were observed microscopically (Carl Zeiss Inc., Germany) at 200× magnification. H&E staining of the paraffin sections demonstrated that the adipocytes were greater in size in the HFD group than the ND group.

The procedure regarding biochemical characterization is as follows. The serum triglyceride (TG), total cholesterol (TC), high-density lipoprotein-cholesterol (HDL-C), glutamic oxaloacetic transaminase (GOT), glutamic pyruvic transaminase (GPT), albumin (ALB), uric acid (UA), and creatinine (CRE) levels were measured using enzymatic assay kits with a FUJI DRI-CHEM analyzer (Fujifilm, Tokyo, Japan). The LDL-C level was calculated as [(TC)-(HDL-C)-(TG/5)].

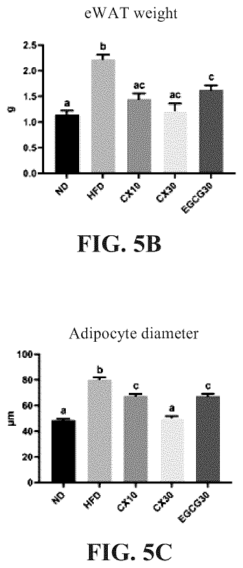

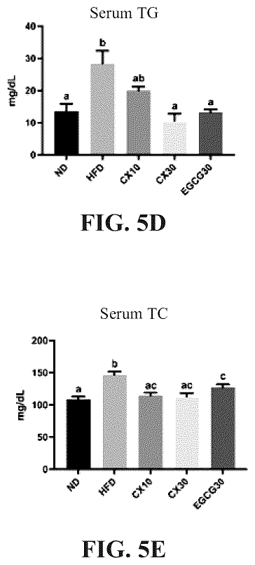

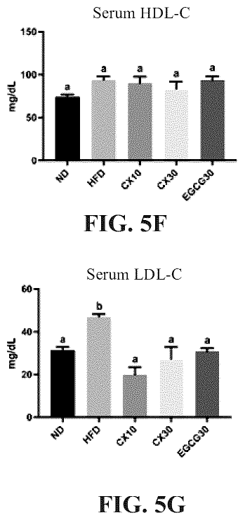

FIGS. 5A-5G show the effect of CX on fat deposition and serum lipid levels in HFD-induced obese mice. (5A) Hematoxylin and eosin (H&E) staining of adipocytes in the epididymal white adipose tissue (eWAT). (5B) The weight of eWAT. (5C) The adipocyte diameters. The level of serum (5D) triglyceride (TG), (5E) total cholesterol (TC), (5F) high-density lipoprotein-cholesterol (HDL-C), and (5G) low-density lipoprotein-cholesterol (LDL-C). The scale bar is 100 μM. Data are shown as means ±SEM. Graph bars labeled with different letters above represent statistically significant results (P<0.05) based on Tukey post hoc tests one-way ANOVA analysis, whereas bars with the same letter correspond to results that show no statistically significant differences. In the case where two letters are present above the bars, each letter should be compared separately with the letters of the other bars to determine whether the results show statistically significant differences.

The sizes of the adipocytes after CX or EGCG administration were similar to the ND group (FIG. 5A). As shown in FIG. 5B, the HFD-induced increase in the mass of eWAT was ameliorated by CX or EGCG intervention. In addition, we found that, compared with the adipocytes from the HFD group, the CX and EGCG groups had lower cell diameters and smaller cell size (FIG. 5C). The level of serum triglyceride (TG) and total cholesterol (TC) in the HFD group were significantly higher than the ND group (FIGS. 5D and 5E). After treatment with CX or EGCG, the level of serum TG and TC were significantly lower than the HFD group. The level of HDL cholesterol (HDL-C) did not differ significantly from the HFD group after CX or EGCG intervention (FIG. 5F). The level of LDL-C was also significantly higher in the HFD group than the ND group but this was somewhat ameliorated by CX or EGCG supplementation (FIG. 5G). This suggests that CX may decrease fat deposition and inhibit hypertriglyceridemia and high cholesterol levels in the HFD mouse model.

### Example 6

### CX Improved Glucose Homeostasis and Insulin Resistance in HFD-Induced Obese Mice

It has been shown that NAFLD is closely associated with insulin resistance, as 70%-80% of obese and diabetic patients have NAFLD. Obesity is a major risk factor for insulin resistance. Insulin resistance causes liver cells to convert glycogen into glucose and release glucose into the blood, reducing the role of fat and muscle in absorbing glucose. In addition, insulin resistance also inhibits β-oxidation of free fatty acids, further promoting hepatic fat accumulation. In this example, in addition to measuring the fasting blood glucose of mice every two weeks, the amount of insulin in the serum of mice was also analyzed after sacrifice. First, we measured the fasting blood glucose, which has a direct relationship with impaired insulin sensitivity.

The procedure regarding blood glucose and intraperitoneal glucose tolerance test (IPGTT) is as follows. After a 16 h overnight fasting period, whole-blood glucose was measured with a glucose analyzer (EASYTOUCH, Taiwan). An enzymatic assay was used to measure the serum insulin concentration (Cisbio, USA). Intraperitoneal glucose tolerance tests (IPGTTs) were performed in all mice 12 weeks after the start of the study. Mice fasted for 16 h were injected intraperitoneally with glucose 1.0 g/kg body weight, blood glucose levels were measured in tail vain blood at 0, 30, 60, 90, 120 and 150 min.

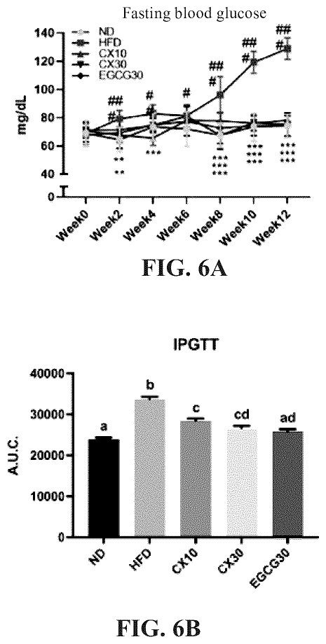

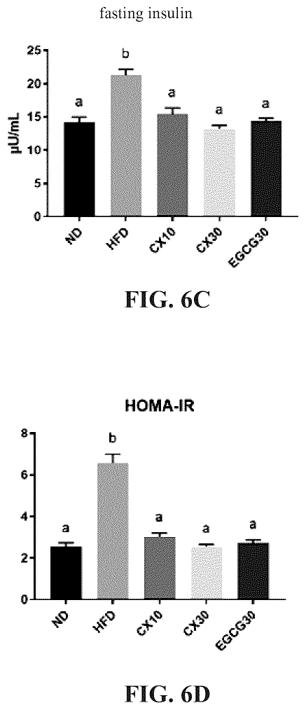

FIGS. 6A and 6B show the effect of CX on glucose metabolism in HFD-induced obese mice. (6A) Fasting blood glucose levels from 0-12 weeks after drug intervention. (6B) Area under the curve (AUC) of IPGTT. Data are shown as means ±SEM. Fasting glucose differences in FIG. 6A were analyzed using unpaired one-tailed Student's t-test (ND vs. HFD, *** p<0.001, HFD vs. drug, *** p<0.001). Graph bars labeled with different letters above represent statistically significant results (P<0.05) based on Tukey post hoc tests one-way ANOVA analysis, whereas bars with the same letter correspond to results that show no statistically significant differences. In the case where two letters are present above the bars, each letter should be compared separately with the letters of the other bars to determine whether the results show statistically significant differences.

First, during the feeding process of mice, blood was drawn every two weeks to analyze fasting blood glucose levels. The fasting blood glucose of the HFD group was significantly higher than that of the ND group starting from the second week, and increased sharply after the eighth week, and was significantly higher than that of the control ND group (p<0.001). The fasting blood glucose in the CX10 group, CX30 group and EGCG30 group has been stable and significantly lower than the HFD group since the second week (FIG. 6A), and extremely significant differences can be observed from the eighth to the twelfth week (p<0.001). In addition, a glucose tolerance test was performed at the 12th week, and blood glucose changes at 0, 30, 60, 90, 120, and 150 minutes were measured and the area under the curve (AUC) was calculated. The AUC of IPGTT was significantly higher in the HFD group than in the ND group, indicating that intraperitoneal injection of glucose could not be effectively removed. Compared with the HFD group, the AUC of both the CX10 group and the CX30 group were significantly decreased, indicating that the glucose tolerance of the drug treatment group was better than that of the HFD group (FIG. 6B). In terms of insulin expression, the HFD group was significantly higher than the ND group, while the insulin expression of the CX10 group and CX30 group was significantly lower than that of the HFD group (FIG. 6C). Next, the insulin resistance index HOMA-IR was calculated and analyzed through blood glucose and insulin amount. It was also observed that the insulin resistance index of the HFD group was significantly higher than that of the ND group. Compared with the HFD group, the indices of both the CX10 group and the CX30 group were significantly lower (FIG. 6D). To summarize the above experimental results, HFD not only causes hyperglycemia in mice and reduces the ability to metabolize glucose, but also causes insulin resistance. The above phenomena were significantly improved in the group given CX treatment.

### Example 7

### CX Ameliorated the Severity of NAFLD in HFD-Induced Obese Mice

One of the distinctive characteristics of metabolic syndrome is NAFLD, which is characterized by triglyceride accumulation in the hepatocytes. To examine the effect of CX on lipid deposition in liver, we weighed the livers and measured the hepatic triglyceride and cholesterol levels of the mice.

The procedure regarding triglyceride and cholesterol analysis of liver tissue is as follows. For triglyceride and cholesterol determinations, mouse liver tissues were extracted and analyzed using triglyceride and cholesterol quantitation assay kits (Abcam, UK), respectively, according to the manufacturer's instruction.

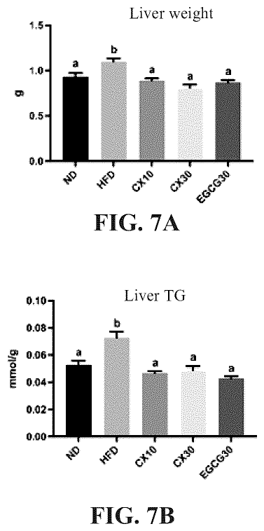

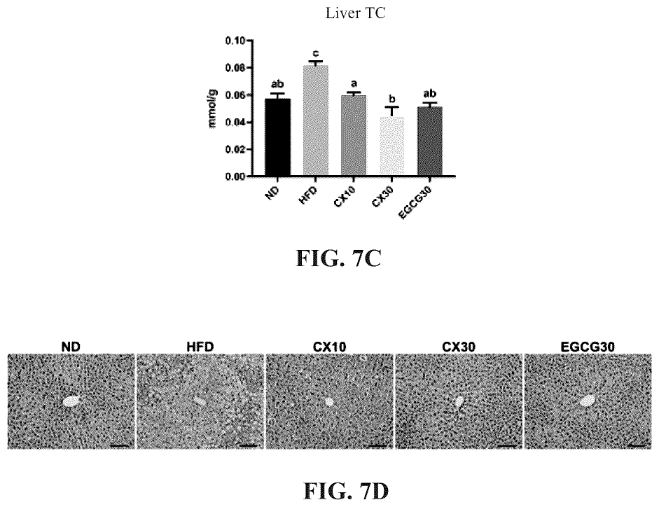

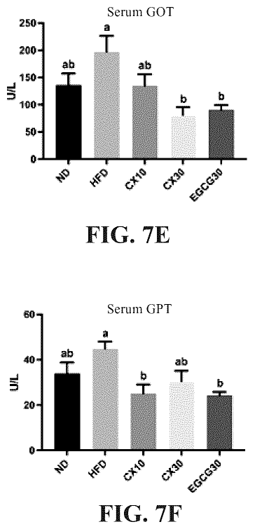

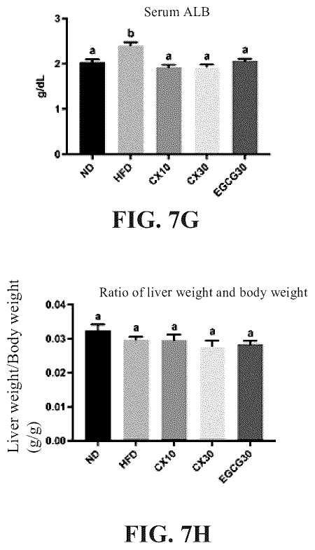

FIGS. 7A-7H show the effect of CX on fat deposition and inflammation in the livers of HFD-induced obese mice. (7A) Liver weight. (7B) Hepatic triglyceride level. (7C) Hepatic cholesterol level. (7D) H&E staining of transverse liver sections (original magnification×200). (7E) Glutamic oxaloacetic transaminase, GOT and (7F) Glutamic-pyruvic transaminase, GPT. Serum GOT and GPT levels as indicators of liver damage. (7G) Serum albumin, ALB, as a liver biochemical indicator. (7H) Ratio of liver weight and body weight. The scale bar is 100 μM. Graph bars labeled with different letters above represent statistically significant results (P<0.05) based on Tukey post hoc tests one-way ANOVA analysis, whereas bars with the same letter correspond to results that show no statistically significant differences. In the case where two letters are present above the bars, each letter should be compared separately with the letters of the other bars to determine whether the results show statistically significant differences.

The result indicated the liver weights of the CX and EGCG groups were significantly lower than HFD group after drugs intervention (FIG. 7A). Moreover, the level of hepatic triglyceride (TG) and cholesterol (TC) was higher after feeding an HFD but much lower after CX and EGCG treatment (FIGS. 7B and 7C). H&E and Oil Red O staining showed considerable lipid deposition in the liver tissue in the HFD group, whereas lipid accumulation was greatly ameliorated and normal morphology of liver cells was maintained by treatment with CX or EGCG (FIG. 7D). Next, we investigated liver damage markers, including glutamic-pyruvic transaminase (GPT) and glutamic oxaloacetic transaminase (GOT). The levels of serum GOT and GPT were higher in HFD mice but these high levels were significantly prevented by CX at both supplementations, indicating an amelioration of liver damage (FIGS. 7E and 7F). Furthermore, the level of albumin, a clinical biochemical marker produced by the liver, was significantly higher in HFD group. The level of albumin had no difference among ND group, CX and EGCG groups (FIG. 7G), suggesting that CX can maintain normal biochemical metabolism of the liver. Ratio of liver weight and body weight did not differ among all groups (FIG. 7H). These results suggested that the severity of HFD-induced NAFLD can be ameliorated, normal liver function can be maintained, and liver hypertrophy can be prevented by CX.

### Example 8

### CX Ameliorated HFD-Induced Renal Damage

Studies have shown that high-fat diet promote renal impairment and cause kidney damage. Therefore, we studied the common biochemical indices of renal function, including creatinine (CRE) and uric acid (UA).

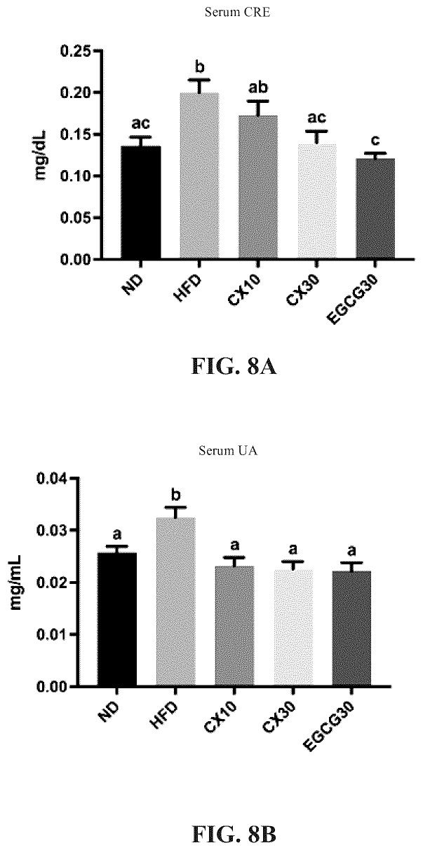

FIGS. 8A and 8B show the effect of CX on renal function of HFD-induced obese mice. The serum levels of the kidney function markers (8A) creatinine, CRE and (8B) uric acid, UA. Creatinine (CRE) is the breakdown product of creatine in human muscles and is filtered and excreted by the renal glomeruli. When the filtration rate of renal glomeruli decreases, CRE in the blood would increase. Uric acid (UA) is a metabolite of purine. High concentrations represent low metabolic function of the kidneys. Therefore, the levels of CRE and UA in the serum are indicators of adverse kidney function. Data are shown as means ±SEM. Graph bars labeled with different letters above represent statistically significant results (P<0.05) based on Tukey post hoc tests one-way ANOVA analysis, whereas bars with the same letter correspond to results that show no statistically significant differences. In the case where two letters are present above the bars, each letter should be compared separately with the letters of the other bars to determine whether the results show statistically significant differences.

The natural compound EGCG has been previously shown to prevent renal impairment and other forms of kidney damage. Therefore, we used EGCG as a reference to compare the effects of CX on protecting renal damage. The levels of creatinine (CRE) and uric acid (UA) were markedly elevated in the high-fat diet (HFD) group compared to the normal diet (ND) group, indicating that a high-fat diet can induce renal damage. Interestingly, CRE and UA levels decreased in CX treatment indicating renal functions were improved by CX (FIGS. 8A and 8B). There was no significant difference in CRE level between CX30 group and ND group and EGCG group. There was no significant difference in uric acid (UA) levels between the CX10-30 group, the ND group, and the EGCG group. This suggests that CX has the potential to alleviate renal damage caused by a high-fat diet (HFD).

In summary, the 6-MBOA of the present invention has been proven that it can effectively modulate lipid metabolism, ameliorate non-alcoholic fatty liver disease, avoid excessive secretion of insulin, and ameliorate insulin resistance and metabolic syndrome through various efficacy experiments.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for modulating lipid metabolism, comprising administering to a subject in need thereof a composition comprising an effective amount of 6-methoxybenzoxazolinone (6-MBOA).

2. The method according to claim 1, wherein the 6-MBOA downregulates de novo lipogenesis in the subject.

3. The method according to claim 1, wherein the 6-MBOA upregulates lipolysis and fatty acid beta-oxidation in the subject.

4. The method according to claim 1, wherein the 6-MBOA produces no concomitant cytotoxicity to immortalized primary human hepatocytes at 25-400 μM.

5. The method according to claim 1, wherein the 6-MBOA mitigates lipid accumulation in the subject.

6. The method according to claim 1, wherein the 6-MBOA increases adenosine 5′-monophosphate-activated protein kinase (AMPK) phosphorylation in the subject.

7. The method according to claim 1, wherein the lipid metabolism is modulated by regulating acetyl-CoA carboxylase (ACC) phosphorylation, and the 6-MBOA simultaneously downregulates expression level of fatty acid synthase (FAS), and upregulates expression levels of adipose triglyceride lipase (ATGL) and carnitine palmitoyl-transferase 1 (CPT1).

8. The method according to claim 1, wherein the composition is a pharmaceutical composition, a food composition, or an external product composition.

9. The method according to claim 8, wherein the pharmaceutical composition is in a dosage form for oral administration.

10. A method for improving non-alcoholic fatty liver disease (NAFLD) and metabolic syndrome, comprising administering to a subject in need thereof a composition comprising an effective amount of 6-methoxybenzoxazolinone (6-MBOA).

11. The method according to claim 10, wherein the 6-MBOA prevents body weight gain in the subject.

12. The method according to claim 10, wherein the 6-MBOA ameliorates adipose tissue hypertrophy and dyslipidemia in the subject.

13. The method according to claim 10, wherein the 6-MBOA improves glucose homeostasis, glucose tolerance, and insulin resistance (HOMA-IR) in the subject.

14. The method according to claim 10, wherein the 6-MBOA improves high fat diet (HFD)-induced fat deposition and inflammation in liver.

15. The method according to claim 10, wherein the 6-MBOA ameliorates HFD-induced renal damage.

16. The method according to claim 10, wherein the composition is a pharmaceutical composition, a food composition, or an external product composition.

17. The method according to claim 16, wherein the pharmaceutical composition is in a dosage form for oral administration.

18. The method according to claim 16, wherein the pharmaceutical composition is in a dosage form for parenteral administration.

19. The method according to claim 16, wherein the pharmaceutical composition further comprises a pharmaceutically acceptable excipient, carrier, auxiliary and/or food additive.

20. The method according to claim 10, wherein the composition has a dosage form of powder, granule, solution, gel, or paste.
